(19) United States
(12) Patent Application Publication
(10) Pub. No.: US 2020/0297763 A1
(43) Pub. Date: Sep. 24, 2020

(54) PLURIPOTENT STEM CELL-DERIVED MACROPHAGE CAPABLE OF TARGETING TUMOR CELLS AND PREPARATION METHOD THEREOF

(71) Applicant: ZHEJIANG UNIVERSITY, Hangzhou (CN)

(72) Inventors: Jin ZHANG, Hangzhou (CN); Li ZHANG, Hangzhou (CN); Lin TIAN, Hangzhou (CN); Tao LUO, Hangzhou (CN)

(21) Appl. No.: 16/892,156

(22) Filed: Jun. 3, 2020

Related U.S. Application Data
(63) Continuation-in-part of application No. PCT/CN2019/099680, filed on Aug. 7, 2019.

(30) Foreign Application Priority Data
Oct. 18, 2018 (CN) 201811218443.2

(51) Int. Cl. A61K 35/15; A61P 35/02; C12N 5/074

(52) U.S. Cl. CPC A61K 35/15; C12N 5/0696; A61P 35/02

(57) ABSTRACT

A macrophage capable of targeting tumor cells and a preparation method thereof are provided. The macrophage comprises a chimeric antigen receptor. The chimeric antigen receptor is expressed on the macrophage which infiltrates more efficiently into tumor than T cells.

### CROSS-REFERENCE TO RELATED APPLICATIONS

The present disclosure is a continuation-in-part of International Application No. PCT/CN2019/099680, filed Aug. 7, 2019, which claims priority to Chinese patent application No. 201811218443.2, filed with the Chinese Patent Office on Oct. 18, 2018 and entitled “Macrophage Capable of Targeting Tumor Cells and Preparation Method Thereof”, which applications are incorporated herein by reference in their entireties.

### BACKGROUND

### (1) Field of the Invention

The present disclosure relates to the field of biotechnology, and particularly to a pluripotent stem cell-derived macrophage capable of targeting tumor cells and a preparation method thereof.

### (2) Related Art

With the development of immunology, and genome editing and synthetic biology, the study of tumor immunotherapy advances rapidly, especially the adoptive immunotherapy which has a broad prospect. Adoptive immunotherapy is a method to treat tumors by adoptive transfusion of lymphocytes cultured in vitro under stimulation back into tumor patients. Chimeric antigen receptor (CAR) modifying T cells is a new method of adoptive immunotherapy for tumors that has developed rapidly in recent years. The CAR modification enables T cells to have better tumor targeting property, stronger killing activity and lasting vitality, which improves the therapeutic effect.

However, on the one hand, engineering in CAR-T cells all faces the problems of low transformation efficiency of vectors and low efficiency of gene editing, and the amplification ability of the engineered cells may not necessarily meet the clinically required cell dose. Hence, one great challenge in the promotion of CAR-T cell therapy is the extremely high cost. Kymriah, the earliest approved CAR-T product of Norvatis in the United States for the treatment of refractory recurrent B-cell leukemia, costs $475,000, reflecting the high cost of this allogeneic individualized cell product from cell collection, virus/CAR-T cell preparation to retransfusion. Moreover, the cells produced in CAR-T cell therapy are limited, and one CAR-T cell cannot be used in the treatment of multiple patients. If gene editing and in vitro amplification are performed on the allogeneic T cells, the obtained correctly edited cells are limited, and immunological rejection is also a technical problem to be solved. On the other hand, a tumor, particularly a solid tumor, has a complex microenvironment inside, including not only tumor cells themselves and T cells, but also macrophages, fibroblasts, etc. The complex solid tumor microenvironment limits the contact of CAR-T cells with tumor cells, and even if CAR-T cells enter the solid tumor, many types of cells will inhibit the effect of CAR-T cells in killing tumor cells, which further promotes the occurrence and development of the tumor and weakens the killing effect of the CAR-T cells. Therefore, it is very necessary to provide a general-purpose product that can be used for allogenic individual and enables obtaining of a large number of finished products capable of efficiently targeting tumor cells at a low cost. In view of this, the present disclosure is proposed.

### SUMMARY

The objects of the present disclosure include, for example, providing a macrophage capable of targeting tumor cells, so as to alleviate the technical problem in the prior art that in the CAR-T cell therapy, CAR-T cells have poor recognition ability and weak killing effect on tumor cells, especially solid tumor cells.

The objects of the present disclosure include, for example, providing a pluripotent stem cell capable of differentiating into the macrophage.

The objects of the present disclosure include, for example, providing a preparation method of a macrophage capable of targeting tumor cells, so as to alleviate the technical problem of lacking a product capable of efficiently targeting tumor cells in the prior art.

The present disclosure provides a macrophage capable of targeting tumor cells, the macrophage comprising a chimeric antigen receptor.

In one or more embodiments, the macrophage is an HLA-I deficient macrophage.

In one or more embodiments, the macrophage is a B2M gene-deficient macrophage.

In one or more embodiments, the macrophage is obtained by directed differentiation of a pluripotent stem cell containing a gene encoding the chimeric antigen receptor.

In one or more embodiments, the pluripotent stem cell is an HLA-I deficient pluripotent stem cell.

In one or more embodiments, the pluripotent stem cell is a B2M gene-deficient pluripotent stem cell.

In one or more embodiments, the pluripotent stem cell comprises an induced pluripotent stem cell and/or an embryonic stem cell.

In one or more embodiments, the gene encoding the chimeric antigen receptor is located on a vector.

In one or more embodiments, the vector comprises a plasmid vector or a viral vector. In one or more embodiments, the viral vector is a retroviral vector, preferably a lentiviral vector.

In one or more embodiments, a plasmid vector used to construct a B2M gene-deficient type is one of the vectors in the following a) or b):

a) capable of expressing gRNA and Cas9 protein;

b) capable of expressing gRNA and Cpf1 protein.

In one or more embodiments, the chimeric antigen receptor comprises an extracellular antigen binding region, a transmembrane region, a costimulatory domain, and an intracellular signal transduction region. In one or more embodiments, the extracellular antigen binding region comprises an sc-Fv, Fab, scFab, or scIgG antibody fragment; and/or the transmembrane region comprises at least one of CD3ζ, CD4, CD8 and CD28; and/or the costimulatory domain comprises at least one of ligands specifically binding to CD27, CD28, CD137, OX40, CD30, CD40, PD-1, LFA-1, CD2, CD7, Lck, DAP10, ICOS, LIGHT, NKG2C, B7-H3, or CD3ζ; and/or the intracellular signal transduction region comprises at least one of CD3ζ, FcεRlγ, PKCθ or ZAP70.

In one or more embodiments, the chimeric antigen receptor further comprises a reporter gene. In one or more embodiments, the reporter gene is a fluorescent reporter gene. In one or more embodiments, the fluorescent reporter gene is any one selected from GFP, EGFP, RFP, mCherry, mStrawberry, Luciferase, mApple, mRuby and EosFP.

In one or more embodiments, the extracellular antigen binding region specifically binds to CD19.

The present disclosure also provides a pluripotent stem cell capable of differentiating into the macrophage described herein.

The present disclosure also provides a preparation method of the macrophage, comprising expressing a gene encoding a chimeric antigen receptor on the macrophage to obtain the macrophage capable of targeting tumor cells.

In one or more embodiments, the preparation method further comprises a step of preparing an HLA-I gene-deficient macrophage; preferably, the preparation method further comprises a step of preparing a B2M gene-deficient macrophage.

In one or more embodiments, the preparation method comprises directed differentiation of a pluripotent stem cell into a macrophage capable of targeting tumor cells, the pluripotent stem cell containing a gene encoding a chimeric antigen receptor.

In one or more embodiments, the pluripotent stem cell is an HLA-I deficient pluripotent stem cell.

In one or more embodiments, the pluripotent stem cell is a B2M gene-deficient pluripotent stem cell.

In one or more embodiments, the pluripotent stem cell comprises an induced pluripotent stem cell and/or an embryonic stem cell.

In one or more embodiments, the gene encoding the chimeric antigen receptor is recombined on a vector and expressed in the macrophage.

In one or more embodiments, a reporter gene is recombined with the chimeric antigen receptor and then ligated to a vector.

In one or more embodiments, the reporter gene is a fluorescent reporter gene. In one or more embodiments, the fluorescent reporter gene is any one selected from GFP, EGFP, RFP, mCherry, mStrawberry, Luciferase, mApple, mRuby and EosFP.

In one or more embodiments, the directed differentiation comprises the steps of: placing an embryoid body resulting from induced differentiation of a pluripotent stem cell in a first medium for a first stage culture, and then in a second medium for a second stage culture, in a third medium for a third stage culture, in a fourth medium for a fourth stage culture, in a fifth medium for a fifth stage culture, in a sixth medium for a sixth stage culture, and in a seventh medium for a seventh stage culture sequentially; the first stage is days 0-1 after inoculation, the second stage is days 2-7 after inoculation, the third stage is days 8-10 after inoculation, the fourth stage is days 10-20 after inoculation, the fifth stage is days 20-22 after inoculation, the sixth stage is days 22-28 after inoculation, and the seventh stage is day 29 after inoculation.

In one or more embodiments, a matrix gel needs to be provided in the culture of the fourth stage, the fifth stage, the sixth stage, and the seventh stage. In one or more embodiments, the matrix gel comprises Matrigel or Laminin-521.

In one or more embodiments, the step of induced differentiation of the pluripotent stem cell into an embryoid body comprises: adding a cell dissociation solution (e.g., a natural enzyme mixture with proteolytic and collagenolytic enzyme activity sold under the tradename Accutase) to the pluripotent stem cell, and incubating the pluripotent stem cell at 36-38° C. for 10-14 h to obtain an embryoid body.

In one or more embodiments, the pluripotent stem cell is treated with Rho-associated, coiled-coil containing protein kinase (ROCK) inhibitor Y27632, before being added with the cell dissociation solution (e.g., Accutase), and incubated at 36-38° C. for 10-14 h to obtain an embryoid body.

In one or more embodiments, the first medium comprises a first basal medium and a first cytokine comprising BMP4 and bFGF; the second medium comprises the first basal medium and a second cytokine comprising BMP4, bFGF, VEGF and SCF; the third medium comprises the first basal medium and a third cytokine comprising bFGF, VEGF, SCF, IGF1, IL-3, M-CSF and GM-CSF; the fourth medium comprises a second basal medium and the third cytokine; the fifth medium comprises the second basal medium and a fourth cytokine comprising bFGF, VEGF, SCF, IGF1, IL-3, M-CSF and GM-CSF; the sixth medium comprises the second basal medium and a fifth cytokine comprising bFGF, VEGF, SCF, 1GF1, M-CSF and GM-CSF; the seventh medium comprises a third basal medium, a sixth cytokine and FBS, the sixth cytokine comprising M-CSF and GM-CSF; wherein the first basal medium and the second basal medium are serum-free mediums; the third basal medium is a serum-containing medium.

In one or more embodiments, the first basal medium is sold under the tradename STEMdiff™ APEL™ 2 or mTeSR1. In one or more embodiments, the second basal medium is sold under the tradename StemPro™-34. In one or more embodiments, the third basal medium is RPMI-1640.

Further provided is use of the macrophage capable of targeting tumor cells according to the present disclosure in the prevention or treatment of a tumor.

In one or more embodiments, the tumor includes at least one of acute lymphoblastic leukemia, acute myelogenous leukemia, cholangiocarcinoma, breast cancer, cervical cancer, chronic lymphocytic leukemia, chronic myelogenous leukemia, colorectal cancer, endometrial cancer, esophageal cancer, gastric cancer, head and neck cancer, Hodgkin's lymphoma, lung cancer, medullary thyroid carcinoma, non-Hodgkin's lymphoma, multiple myeloma, kidney cancer, ovarian cancer, pancreatic cancer, neuroglioma, melanoma, liver cancer, prostate cancer and urinary bladder cancer.

Further provided is a method for preventing or treating a tumor, comprising administering the macrophage capable of targeting tumor cells of the present disclosure to a subject in need thereof.

In one or more embodiments, the tumor includes at least one of acute lymphoblastic leukemia, acute myelogenous leukemia, cholangiocarcinoma, breast cancer, cervical cancer, chronic lymphocytic leukemia, chronic myelogenous leukemia, colorectal cancer, endometrial cancer, esophageal cancer, gastric cancer, head and neck cancer, Hodgkin's lymphoma, lung cancer, medullary thyroid carcinoma, non-Hodgkin's lymphoma, multiple myeloma, kidney cancer, ovarian cancer, pancreatic cancer, neuroglioma, melanoma, liver cancer, prostate cancer and urinary bladder cancer.

Compared with the prior art, the advantageous effects of the present disclosure include at least as follows.

The present disclosure provides a macrophage capable of targeting tumor cells, the macrophage containing a chimeric antigen receptor. The inventors have found that the CAR-T cell therapy has some technical defects in the treatment of tumors, i.e., due to the limitation of the microenvironment of a solid tumor, it is very difficult for CAR-T cells to enter the tumor, and even if the CAR-T cells enter the tumor, the effect of killing tumor cells thereof is weakened due to the inhibition in the microenvironment. In view of the above technical defects, the inventors have proposed another idea of tumor immunotherapy in which a chimeric antigen receptor is expressed in the macrophage. Compared with T cells, the macrophage has the advantages of being easier to enter the solid tumor and less likely to be inhibited by other types of cells, and therefore can play a better role in tumor immunotherapy. Since the expressed chimeric antigen receptor is located on the surface of the macrophage, the macrophage can accurately target tumor cells. Moreover, the inventors have found through experiments that the chimeric antigen receptor suitable for T cells is also suitable for the macrophage, that is, the application of the chimeric antigen receptor in the CAR-T cell therapy to the macrophage can realize expressing the chimeric antigen receptor on the surface of the macrophage, targeting tumor cells and activating the macrophage to phagocytize tumor cells. Therefore, the discovery of using a chimeric antigen receptor to modify a macrophage provides a new idea and technical means for solid tumor immunotherapy, which is of great significance for tumor immunotherapy.

The present disclosure provides a preparation method of the macrophage capable of targeting tumor cells, which provides a whole new idea for tumor immunotherapy.

### BRIEF DESCRIPTION OF DRAWINGS

FIG. 1A is a graph showing the results of flow cytometry detection of a marker CD45 in a myeloid cell on day 14 in Example 9 of the present disclosure;

FIG. 1B is a graph showing the results of flow cytometry detection of a marker CD34 in the myeloid cell on day 14 in Example 9 of the present disclosure;

FIG. 1C is a graph showing the results of flow cytometry detection of a marker CD11b in the myeloid cell on day 14 in Example 9 of the present disclosure;

FIG. 1D is a graph showing the results of flow cytometry detection of a marker CD14 in the myeloid cell on day 14 in Example 9 of the present disclosure;

FIG. 1E is a graph showing the results of flow cytometry detection of a marker CD11b in a mature macrophage on day 45 in Example 9 of the present disclosure;

FIG. 1F is a graph showing the results of flow cytometry detection of a marker CD14 in the mature macrophage on day 45 in Example 9 of the present disclosure;

FIG. 1G is a graph showing the results of flow cytometry detection of a marker CD163 in the mature macrophage on day 45 in Example 9 of the present disclosure;

FIG. 1H is a graph showing the results of flow cytometry detection of a marker CD86 in the mature macrophage on day 45 in Example 9 of the present disclosure;

FIG. 2A shows the expression of a chimeric antigen receptor on the surface of a wild-type iPS cell, detected by flow cytometry, in Example 10 of the present disclosure;

FIG. 2B shows the expression of the chimeric antigen receptor on the surface of an iPS cell stably expressing the chimeric antigen receptor, detected by flow cytometry, in Example 10 of the present disclosure;

FIG. 2C shows the expression of the chimeric antigen receptor on the cell surface on a macrophage into which the iPS cell stably expressing the chimeric antigen receptor differentiated, detected by flow cytometry, in Example 10 of the present disclosure;

FIG. 3 is a graph showing the results of cell immune test after B2M knockout, detected by flow cytometry, in Example 11 of the present disclosure;

FIG. 4 is a focusing microscope photograph showing a macrophage resulting from iPS differentiation phagocytizing Raji cancer cells in Example 12 of the present disclosure.

FIG. 5A shows co-culture of 2×10\hat{ }5 macrophages iMAC obtained by differentiation of iPS or ES cells overexpressing the chimeric antigen receptor CD19 CAR with 4×10\hat{ }4 CD19 antigen-expressing K562 tumor cells and non-CD19 antigen-expressing K562 cells for 24 hours separately to detect the phagocytosis of the iMAC for tumor cells.

FIG. 5B shows, based on the experiment of FIG. 5A, extraction of RNA from the macrophages that have been co-cultured with tumor cells, to detect the expressions of cytokines (TNF, IL-6 and IL-1 beta).

### DETAILED DESCRIPTION

The embodiments of the present disclosure will be described in detail below in connection with examples, but it will be understood by those skilled in the art that the following examples are merely illustrative of the present disclosure and should not be construed as limiting the scope of the present disclosure. Examples are carried out in accordance with conventional conditions or conditions recommended by the manufacturer if no specific conditions are specified in the examples.

A macrophage capable of targeting tumor cells, the macrophage comprising a chimeric antigen receptor.

The inventors have found that the CAR-T cell therapy has some technical defects in the treatment of tumors, i.e., due to the limitation of the microenvironment of a solid tumor, it is very difficult for CAR-T cells to enter the tumor, and even if the CAR-T cells enter the tumor, the effect of killing tumor cells thereof is weakened due to the inhibition in the microenvironment. In view of the technical defects, the inventors have proposed another idea of tumor immunotherapy in which a chimeric antigen receptor is expressed in the macrophage. Compared with T cells, the macrophage has the advantages of being easier to enter the solid tumor and less likely to be inhibited by other types of cells, and therefore can play a better role in tumor immunotherapy. Since the expressed chimeric antigen receptor is located on the surface of the macrophage, the macrophage can accurately target tumor cells. Moreover, the inventors have found through experiments that the chimeric antigen receptor suitable for T cells is also suitable for the macrophage, that is, the application of the chimeric antigen receptor in the CAR-T cell therapy to the macrophage can realize expressing the chimeric antigen receptor on the surface of the macrophage, targeting tumor cells and activating the macrophage to phagocytize tumor cells. Therefore, the discovery of using a chimeric antigen receptor to modify a macrophage provides a new idea and technical means for solid tumor immunotherapy, which is of great significance for tumor immunotherapy.

In one or more embodiments, the macrophage is an HLA-I (human lymphocyte antigen I) deficient macrophage. Making the macrophage express a chimeric antigen receptor enables the macrophage to efficiently target tumor cells and enables itself to be activated for phagocytosis of tumor cells. However, due to the specific recognition effect of MHC (major histocompatibility complex), there occur immunological rejection in allogeneic cell transplantation and graft versus host reaction. Thus, the universality of the macrophage capable of targeting tumor cells needs to be improved. By modifying the MHC of the macrophage to construct an HLA-I gene deficient cell, it is possible to avoid allogeneic rejection, improve the universality of the macrophage that can target tumor cells, and further reduce the cost of tumor immunotherapy. HLA-I is an allogenic antigen with high polymorphism, which is closely related to organ transplantation, immunological rejection, etc. HLA-I of a macrophage can be knocked out, thereby reducing allogeneic immunological rejection, which has a broader and more general application range, as compared with the wild-type CAR-T cells, etc. that are currently used for immune cell therapy. The method employed is to directly knock out the B2M gene from the HLA-I complex, to achieve reduction in immunogenicity of the cells so as to avoid rejection of the host to the transplanted cells after differentiation into immune cells, thereby realizing allotransplantation.

In one or more embodiments, the macrophage is B2M gene-deficient macrophage. B2M, i.e., β2 microglobulin, is a member of the MHC class I molecules, which is present in all nucleated cells, except red blood cells. B2M is essential to the expression of the MHC class I protein on the cell surface and the stability of the peptide binding region. In fact, in the absence of B2M, few MHC class I proteins can be detected on the cell surface. Construction of B2M gene-deficient macrophages can effectively reduce the immunological rejection of the host to the transplanted cells.

In one or more embodiments, the macrophage is obtained by directed differentiation of a pluripotent stem cell containing a gene encoding the chimeric antigen receptor. Both T cells and macrophages are mature cells, and the amplification capacity of the cells is limited. Moreover, due to the impacts of the transformation efficiency of the vector and the gene editing efficiency, the obtained correctly edited cells are very limited, which may not necessarily meet the clinically required cell dose, and the product cost is too high. This problem can be solved by directed differentiation of pluripotent stem cells, preferably genetically engineered single clone of pluripotent stem cells, into macrophages. Moreover, the pluripotent stem cells are genetically modified such that they can express a gene encoding a chimeric antigen receptor and/or become a B2M deficient type, and then the pluripotent stem cells are subjected to directed differentiation to obtain a large number of macrophages capable of targeting tumor cells. Pluripotent stem cells have the ability to proliferate indefinitely and differentiate into immune cells, and after gene editing of the pluripotent stem cells, monoclonal cells that are edited correctly and do not have an off-target effect can be selected.

In one or more embodiments, the pluripotent stem cell is an HLA-I deficient pluripotent stem cell. The pluripotent stem cells are subjected to HLA-I deficient modification to obtain macrophages that have high universality, have no immunological rejection in allotransplantation and can target tumor cells.

In one or more embodiments, the pluripotent stem cell is a B2M gene-deficient pluripotent stem cell.

In one or more embodiments, the pluripotent stem cell comprises an induced pluripotent stem cell and/or an embryonic stem cell.

In one or more embodiments, the gene encoding the chimeric antigen receptor is located on a vector.

In one or more embodiments, the vector comprises a plasmid vector or a viral vector.

In one or more embodiments, the viral vector is a retroviral vector, preferably a lentiviral vector.

In one or more embodiments, a plasmid vector used to construct a B2M gene-deficient type is one of the vectors in the following a) or b): a) capable of expressing gRNA and Cas9 protein; b) capable of expressing gRNA and Cpf1 protein.

In one or more embodiments, the chimeric antigen receptor comprises an extracellular antigen binding region, a transmembrane region, a costimulatory domain, and an intracellular signal transduction region. It should be noted that the chimeric antigen receptor suitable for T cells can be used as a chimeric antigen receptor of the macrophage. In one or more embodiments, the extracellular antigen binding region comprises an sc-Fv, Fab, scFab, or scIgG antibody fragment. In one or more embodiments, the antigen-binding region recognizing tumors recognizes any antigen of a group consisting of CD19, CD20, CD22, CD30, GD2, HER2, CAIX, CD171, Mesothelin, Claudin 18.2, LMP1, EGFR, Muc1, GPC3, EphA2, EpCAM, MG7, CSR, α-fetoprotein (AFP), α-actinin-4, A3, an antigen specific to A33 antibody, ART-4, B7, Ba 733, BAGE, BrE3 antigen, CA125, CAMEL, CAP-1, carbonic anhydrase IX, CASP-8/m, CCL19, CCL21, CD1, CD1a, CD2, CD3, CD4, CD5, CD8, CD11A, CD14, CD15, CD16, CD18, CD21, CD23, CD25, CD29, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD126, CD132, CD133, CD138, CD147, CD154, CDC27, CDK-4/m, CDKN2A, CTLA4, CXCR4, CXCR7, CXCL12, HIF-1α, colon specific antigen p (CSAp), CEA (CEACAM-5), CEACAM-6, c-Met, DAM, EGFRvIII, EGP-1 (TROP-2), EGP-2, ELF2-M, Ep-CAM, a fibroblast growth factor (FGF), Flt-1, Flt-3, a folate receptor, G250 antigen, GAGE, gp100, GRO-β, HLA-DR, HM1.24, human chorionic gonadotropin (HCG) and its subunits, HMGB-1, hypoxia-inducible factor (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor 1 (IGF-1), KC4 antigen, KS-1 antigen, KS1-4, Le-Y, LDR/FUT, macrophage migration inhibitory factor (MIF), MAGE, MAGE-3, MART1, MART-2, NY-ESO-1, TRAG-3, mCRP, MCP-1, MIP-1A, MIP-1B, MIF, MUC2, MUC3, MUC4, MUC5ac, MUC13, MUC16, MUM-1/2, MUM-3, NCA66, NCA95, NCA90, pancreatic cancer mucin, a PD1 receptor, a placental growth factor, p53, PLAGL2, prostatic acid phosphatase, PSA, PRAME, PSMA, PIGF, ILGF, ILGF-1R, IL-6, IL-25, RS5, RANTES, T101, SAGE, S100, survivin, survivin-2B, TAC, TAG-72, tenascin, a TRAIL receptor, TNF-α, Tn antigen, Thomsen-Friedenreich antigen, a tumor necrosis antigen, VEGFR, ED-B fibronectin, WT-1, 17-1A antigen, complement factors C3, C3a, C3b, C5a and C5, an angiogenesis marker, bc1-2, bc1-6, Kras, an oncogene marker and an oncogene product. In one or more embodiments, the extracellular antigen binding region specifically binds to CD19. In one or more embodiments, the transmembrane region comprises at least one of CD3ζ, CD4, CD8 and CD28. In one or more embodiments, the costimulatory domain comprises at least one of ligands specifically binding to CD27, CD28, CD137, OX40, CD30, CD40, PD-1, LFA-1, CD2, CD7, Lck, DAP10, ICOS, LIGHT, NKG2C, B7-H3, or CD3. In one or more embodiments, the intracellular signal transduction region comprises at least one of CD3ζ, FcεRlγ, PKCθ and ZAP70.

In one or more embodiments, the chimeric antigen receptor further comprises a reporter gene. In one or more embodiments, the reporter gene is a fluorescent reporter gene. In one or more embodiments, the fluorescent reporter gene is any one selected from GFP, EGFP, RFP, mCherry, mStrawberry, Luciferase, mApple, mRuby and EosFP.

In one or more embodiments, the macrophage capable of targeting tumor cells or the therapy based on differentiation into the macrophage is suitable for the treatment of cancers. It is contemplated that any type of tumors and any type of tumor antigens can be targeted. Exemplary types of cancers that can be targeted include acute lymphoblastic leukemia, acute myelogenous leukemia, cholangiocarcinoma, breast cancer, cervical cancer, chronic lymphocytic leukemia, chronic myelogenous leukemia, colorectal cancer, endometrial cancer, esophageal cancer, gastric cancer, head and neck cancer, Hodgkin's lymphoma, lung cancer, medullary thyroid carcinoma, non-Hodgkin's lymphoma, multiple myeloma, kidney cancer, ovarian cancer, pancreatic cancer, neuroglioma, melanoma, liver cancer, prostate cancer, urinary bladder cancer, etc. However, it should be noted that those skilled in the art shall appreciate that tumor-associated antigens of any type of cancers are actually known.

A preparation method of the macrophage capable of targeting tumor cells comprises expressing a gene encoding a chimeric antigen receptor in the macrophage to obtain the macrophage capable of targeting tumor cells. This method provides a whole new idea for tumor immunotherapy.

In one or more embodiments, the preparation method further comprises a step of preparing an HLA-I gene-deficient macrophage.

In one or more embodiments, the preparation method further comprises a step of preparing a B2M gene-deficient macrophage.

In one or more embodiments, the preparation method comprises directed differentiation of a pluripotent stem cell into a macrophage capable of targeting tumor cells, the pluripotent stem cell containing a gene encoding a chimeric antigen receptor.

In one or more embodiments, the pluripotent stem cell is an HLA-I deficient pluripotent stem cell.

In one or more embodiments, the pluripotent stem cell is a B2M gene-deficient pluripotent stem cell.

In one or more embodiments, the pluripotent stem cell comprises an induced pluripotent stem cell and/or an embryonic stem cell.

In one or more embodiments, the gene encoding the chimeric antigen receptor is recombined on a vector and expressed in the macrophage.

In one or more embodiments, a reporter gene is recombined with the chimeric antigen receptor and then ligated to the vector.

In one or more embodiments, the reporter gene is a fluorescent reporter gene. In one or more embodiments, the fluorescent reporter gene is any one selected from GFP, EGFP, RFP, mCherry, mStrawberry, Luciferase, mApple, mRuby and EosFP.

In one or more embodiments, the directed differentiation comprises the steps of: placing an embryoid body resulting from induced differentiation of a pluripotent stem cell in a first medium for a first stage culture, and then in a second medium for a second stage culture, in a third medium for a third stage culture, in a fourth medium for a fourth stage culture, in a fifth medium for a fifth stage culture, in a sixth medium for a sixth stage culture, and in a seventh medium for a seventh stage culture sequentially, wherein the first stage is days 0-1 after inoculation, the second stage is days 2-7 after inoculation, the third stage is days 8-10 after inoculation, the fourth stage is days 10-20 after inoculation, the fifth stage is days 20-22 after inoculation, the sixth stage is days 22-28 after inoculation, and the seventh stage is day 29 after inoculation.

In the above-described cell induction and culture method, the pluripotent stem cells with genes encoding chimeric antigen receptors are first cultured to form embryoid bodies which are then cultured in a cell induction medium to finally obtain a large number of macrophages capable of targeting tumor cells.

It should be noted that mesoblastic cells are obtained in the first stage, hematopoietic cells are obtained in the second stage, myeloid cells are obtained in the third stage, and mature macrophages are obtained in the fourth stage.

In one or more embodiments, the second stage culture requires replacement of a new second medium every other day, the third stage culture requires replacement of a new third medium every other day, and the cells to be cultured in the fifth stage are suspension cells obtained after the fourth stage culture.

In one or more embodiments, the process of pluripotent stem cells forming embryoid bodies (EB) is as follows: mTeSR1, DMEM/F12 and Versene solution are preheated to 15-25° C. for cell passage. Y27632 is a Rock kinase inhibitor and used at a concentration of 3 μM.

a) washing wells with 1 ml DPBS;

b) aspirating DPBS, adding 1 ml Versene containing Y27632, and incubating at 37° C. for 4 min;

c) dissociating 1-2 times with a pipette and taking the cells out (in general, better EBs will be formed if the cells are still in larger mass);

d) immediately transferring the cells to a centrifuge tube containing DMEM/F12 to dilute Versene at a ratio of 1:5-9; washing the wells once with 1 ml DMEM/F12, collecting the remaining cells and transferring the same to a test tube for centrifugation at 300×g for 5 min; and

e) resuspending the cells in the mTeSR1 medium containing Y27632 and placing the cells on an ultra-low attachment plate, the segregation ratio being 1-2:1 (90% of pluripotent stem cells per well).

In one or more embodiments, the number of cells on day 10 inoculation of cells is 20-25 cells/ml.

In one or more embodiments, in the solution of the present disclosure, the medium may be replaced in any one of the following manners 1)-3): 1) leaving the cells in a tube for 5 min (the tube is coated with 0.1% BSA in DPBS); 2) centrifuging at 300 rpm/min for 3 min; and 3) filtering by a filter, and replacing the medium.

In one or more embodiments, in the cell induction and culture process, the volumes of the mediums of different types of plates are as follows: 2.0 mL/well for a 6-well plate, 0.5 MI/well for a 24-well plate, and 150 μL/well for a 96-well plate.

In one or more embodiments, a matrix gel needs to be provided in the culture of the fourth stage, the fifth stage, the sixth stage, and the seventh stage.

In one or more embodiments, the matrix gel comprises Matrigel or Lam inin-521.

In one or more embodiments, the step of inducing the pluripotent stem cell to differentiate into an embryoid body comprises: treating the pluripotent stem cell with a Rock kinase inhibitor Y27632, then adding the cell dissociation solution Accutase to the pluripotent stem cell, and incubating the pluripotent stem cell at 36-38° C. for 10-14 h to obtain an embryoid body.

In one or more embodiments, the first medium comprises a first basal medium and a first cytokine comprising BMP4 and bFGF; the second medium comprises the first basal medium and a second cytokine comprising BMP4, bFGF, VEGF and SCF; the third medium comprises the first basal medium and a third cytokine comprising bFGF, VEGF, SCF, IGF1, IL-3, M-CSF and GM-CSF; the fourth medium comprises a second basal medium and the third cytokine; the fifth medium comprises the second basal medium and a fourth cytokine comprising bFGF, VEGF, SCF, IGF1, IL-3, M-CSF and GM-CSF; the sixth medium comprises the second basal medium and a fifth cytokine comprising bFGF, VEGF, SCF, IGF1, M-CSF and GM-CSF; the seventh medium comprises a third basal medium, a sixth cytokine and FBS, the sixth cytokine comprising M-CSF and GM-CSF; wherein the first basal medium and the second basal medium are serum-free mediums; and the third basal medium is a serum-containing medium.

The combinations of the cell induction mediums are used in sequence, so that the embryoid body cells can be rapidly and largely induced to differentiate into macrophages. Since the embryoid bodies are obtained by differentiation of pluripotent stem cells and the pluripotent stem cells can stably express chimeric antigen receptors, the obtained macrophages can express chimeric antigen receptors and have the ability to phagocytize tumor cells. The first six mediums are serum-free mediums, which can provide basic nutrients for cell growth, proliferation and differentiation at various stages while reducing the risk of contamination. In addition, the seventh medium contains serum and FBS, which can effectively maintain the growth of the macrophages. Each of the mediums contains many specific cytokines, and therefore can promote directed differentiation of the cells so as to finally obtain a large number of macrophages with stable performance and high quality.

BMP4 (bone morphogenetic protein 4) belongs to the TGF-β superfamily and plays an important role in the embryonic development and regenerative repair of bone. BMP4 is involved in the regulation of the biological process of cells such as proliferation, differentiation and apoptosis, and plays an important role in embryonic development, environmental stability in tissues and organs after birth and the occurrence of many tumors.

bFGF is a kind of fibroblast growth factors, which is a basic fibroblast growth factor, is an inducing factor of cell morphogenesis and differentiation, and can induce and promote the proliferation and differentiation of many kinds of cells.

VEGF (vascular endothelial growth factor) is a highly specific vascular endothelial growth factor, which has the effects of increasing vascular permeability, promoting migration of vascular endothelial cells and extracellular matrix degeneration, and promoting cell proliferation and angiogenesis.

SCF (stem cell factor) is an acid glycoprotein produced by matrix cells in the bone marrow microenvironment.

IGF1 is a kind of insulin-like growth factors, which promotes cell growth and differentiation.

IL-3 (interleukin-3) is a kind of cytokines of the chemokine family, which can regulate hematopoiesis and immunity.

M-CSF (macrophage CSF) and GM-CSF (granulocyte and macrophage CSF) both belong to colony stimulating factors (CSF). M-CSF has the functions of stimulating macrophage colony and stimulating granulocytes, and lowers blood cholesterol. GM-CSF can stimulate the formation of granulocyte and macrophage colonies and has the function of stimulating granulocytes.

FBS is fetal bovine serum, which is a light yellow, clear, slightly viscous liquid with no hemolysis or foreign bodies. FBS contains the least components harmful to cells, such as antibodies and complements, and contains abundant nutrients essential for cell growth.

In one or more embodiments, the first basal medium is STEMdiff™ APEL™ 2 or mTeSR1.

In one or more embodiments, the second basal medium is StemPro™-34.

In one or more embodiments, the third basal medium is RPMI-1640.

In one or more embodiments, in the first medium, the final concentrations of BMP4 and bFGF are 8-12 ng/ml and 3-7 ng/ml, respectively. The concentration of BMP4 is typically, but not limited to, 8 ng/ml, 10 ng/ml or 12 ng/ml; and the concentration of bFGF is typically, but not limited to, 3 ng/ml, 5 ng/ml or 7 ng/ml.

In one or more embodiments, in the second medium, the final concentrations of BMP4, bFGF, VEGF and SCF are 8-12 ng/ml, 3-7 ng/ml, 48-52 ng/ml and 95-105 ng/ml, respectively. The concentration of BMP4 is typically, but not limited to, 8 ng/ml, 10 ng/ml or 12 ng/ml; the concentration of bFGF is typically, but not limited to, 3 ng/ml, 5 ng/ml or 7 ng/ml; the concentration of VEGF is typically, but not limited to, 48 ng/ml, 50 ng/ml or 52 ng/ml; and the concentration of SCF is typically, but not limited to, 95 ng/ml, 99 ng/ml, 100 ng/ml, 104 ng/ml or 105 ng/ml.

In one or more embodiments, in the third medium, the final concentrations of bFGF, VEGF, SCF, IGF1, IL-3, M-CSF and GM-CSF are 8-12 ng/ml, 48-52 ng/ml, 48-52 ng/ml, 8-12 ng/ml, 23-27 ng/ml, 48-52 ng/ml and 48-52 ng/ml, respectively. The concentration of bFGF is typically, but not limited to, 8 ng/ml, 10 ng/ml or 12 ng/ml; the concentration of VEGF is typically, but not limited to, 48 ng/ml, 50 ng/ml or 52 ng/ml; the concentration of SCF is typically, but not limited to, 48 ng/ml, 50 ng/ml or 52 ng/ml; the concentration of IGF1 is typically, but not limited to, 8 ng/ml, 10 ng/ml or 12 ng/ml; the concentration of IL-3 is typically, but not limited to, 23 ng/ml, 25 ng/ml or 27 ng/ml; the concentration of M-CSF is typically, but not limited to, 48 ng/ml, 50 ng/ml or 52 ng/ml; and the concentration of GM-CSF is typically, but not limited to, 48 ng/ml, 50 ng/ml or 52 ng/ml.

In one or more embodiments, in the fifth medium, the final concentrations of bFGF, VEGF, SCF, IGF1, IL-3, M-CSF and GM-CSF are 8-12 ng/ml, 48-52 ng/ml, 48-52 ng/ml, 8-12 ng/ml, 23-27 ng/ml, 95-105 ng/ml and 95-105 ng/ml, respectively. The concentration of bFGF is typically, but not limited to, 8 ng/ml, 10 ng/ml or 12 ng/ml; the concentration of VEGF is typically, but not limited to, 48 ng/ml, 50 ng/ml or 52 ng/ml; the concentration of SCF is typically, but not limited to, 48 ng/ml, 50 ng/ml or 52 ng/ml; the concentration of IGF1 is typically, but not limited to, 8 ng/ml, 10 ng/ml or 12 ng/ml; the concentration of IL-3 is typically, but not limited to, 23 ng/ml, 25 ng/ml or 27 ng/ml; the concentration of M-CSF is typically, but not limited to, 95 ng/ml, 99 ng/ml, 102 ng/ml, 104 ng/ml or 105 ng/ml; and the concentration of GM-CSF is typically, but not limited to, 95 ng/ml, 99 ng/ml, 102 ng/ml, 104 ng/ml or 105 ng/ml.

In one or more embodiments, in the sixth medium, the final concentrations of bFGF, VEGF, SCF, IGF1, M-CSF and GM-CSF are 8-12 ng/ml, 48-52 ng/ml, 48-52 ng/ml, 8-12 ng/ml, 95-105 ng/ml and 95-105 ng/ml, respectively. The concentration of bFGF is typically, but not limited to, 8 ng/ml, 10 ng/ml or 12 ng/ml; the concentration of VEGF is typically, but not limited to, 48 ng/ml, 50 ng/ml or 52 ng/ml; the concentration of SCF is typically, but not limited to, 48 ng/ml, 50 ng/ml or 52 ng/ml; the concentration of IGF1 is typically, but not limited to, 8 ng/ml, 10 ng/ml or 12 ng/ml; the concentration of M-CSF is typically, but not limited to, 95 ng/ml, 99 ng/ml, 102 ng/ml, 104 ng/ml or 105 ng/ml; and the concentration of GM-CSF is typically, but not limited to, 95 ng/ml, 99 ng/ml, 100 ng/ml, 104 ng/ml or 105 ng/ml.

In some embodiments, in the seventh medium, the final concentrations of FBS, M-CSF and GM-CSF are 8-12% by mass, 95-105 ng/ml and 95-105 ng/ml, respectively. The mass fraction of FBS is typically, but not limited to, 8%, 10% or 12%; the concentration of M-CSF is typically, but not limited to, 95 ng/ml, 99 ng/ml, 100 ng/ml, 104 ng/ml or 105 ng/ml; and the concentration of GM-CSF is typically, but not limited to, 95 ng/ml, 97 ng/ml, 100 ng/ml, 104 ng/ml or 105 ng/ml. In one or more embodiments, FBS in the seventh medium is subjected to an inactivation treatment.

In one or more embodiments, the present disclosure further relates to a pluripotent stem cell that can differentiate into the macrophage capable of targeting tumor cells. The pluripotent stem cell, after gene editing modification, can directed-differentiate into the macrophage under specific culture conditions.

The present disclosure is further described below by specific examples. However, it is to be understood that these examples are merely for the purpose of illustration in more detail, and shall not be construed as limiting the present disclosure in any form.

### EXAMPLES

### Example 1: Preparation of Induced Pluripotent Stem Cells

On day −1, 10 ml of peripheral blood was extracted from a patient or a volunteer, and was subjected to separation by lymphocyte separation solution to obtain PBMCs (peripheral blood mononuclear cells), and the PBMCs were cultured with H3000+CC100 to revive MEF cells (fibroblasts).

On day 0, 1-2 million PBMCs were taken out, and PBMCs were transformed with the plasmids containing reprogramming factors OCT4, SOX2, KLF4, LIN28 and L-MYC by electroporation, the cells after electroporation-based transformation were cultured in H3000+CC100 medium and centrifuged 4 h later at 250 rcf for 5 min, with the supernatant discarded, and then resuspended in the H3000+CC100 medium, and cultured in a MEF cell plate.

On day 2, the MEF cells were revived.

On day 3, the cell supernatant was taken into a 15 ml centrifuge tube, the adherent cells were digested with 200 ul Tryple for 5 min, the digestion was terminated with 1 ml H3000, the cells were then dissociated with pipette and transferred into a corresponding centrifuge tube, centrifuged at 250 rcf for 5 min, with the supernatant discarded, resuspended in the H3000+CC100 medium, and then cultured in a new MEF cell plate.

On day 4, 200 ul E8 medium was added thereto.

On days 6, 8 and 10, 1 ml medium was taken out and centrifuged at 250 rcf for 5 min, with the supernatant discarded, and then cells were resuspended with 1.2 ml E8 medium, and cultured in the original cell plate.

On days 11-20, the supernatant was aspirated, and the medium was replaced with E8 medium.

Colonies appeared on about day 15, and when the cells grew to a certain extent, the monoclonal cells were selected and placed in a Matrigel-containing 96-well plate for continuous culture and passage, to obtain iPS cells (induced pluripotent stem cells).

### Example 2: Reviving, Culture and Passage of 293T Cells

(1) Reviving: The frozen cells were taken out from a liquid nitrogen container and quickly placed in a 37° C. water bath kettle, and were quickly shaken to thaw cells. A 15 ml centrifuge tube was prepared in a super clean bench, 5 ml complete medium and cells in a freezing tube were added thereto, mixed well, and centrifuged at 250 rcf/min for 5 min. The supernatant was discarded, and the resultant mixture was resuspended with 5 ml complete medium and transferred into a T25 culture flask, and cultured in a 5% CO2 incubator at 37° C. The survival rate of the cells was observed the next day, the used medium was discarded and 5 ml fresh medium was added.

(2) Culture and passage: The cells were passaged and cultured when growing to 80%-90%. The supernatant was discarded. 5 ml PBS was added and the cells were shaken gently. PBS was discarded. 1 ml 0.25% tyrisin was added to digest the cells for 10 s to 20 s until the cells became round and the intercellular space became large. 3 ml complete medium was added, and the mixture was mixed well and then transferred to a 15 ml centrifuge tube, and centrifuged at 250 rcf/min for 5 min. The supernatant was discarded. The resultant mixture was resuspended with 2 ml complete medium and transferred into a T75 culture flask in which 13 ml complete medium was reserved, and then cultured as described above.

### Example 3: Construction of Lentiviral Vector

Lenti-EF1a-CD19-T2A-EGFP-Puro, comprises scFv specifically binding to a CD19 antigen, a transmembrane domain from CD8, a costimulatory domain from 4-1BB, and an intracellular domain from CD3ζ, and also carries a fluorescent gene EGFP and a puromycin resistance gene as a screening gene.

### Example 4: Identification of Lentiviral Vector

The vector was identified, by enzyme digestion with the endonucleases EcoRI and XbaI. The results showed that the digested products had correct band size.

### Example 5: Preparation of Lentivirus

When 293T cells grew to 60-70%, transfection of lentiviral expression vectors, packaging vectors and envelope vectors at a ratio of 4:3:1 was carried out mediated by lip2000 in a 10 cm cell culture plate, the liquid was replaced 6 h later, supernatants was collected 24 h later and 48 h later, respectively, the collected supernatant was filtered with a 0.22 um filter membrane, then 1/2 volume of 25% PEG was added, and the mixture was left overnight at 4° C., and centrifuged at 4000 rcf at 4° C. for 20 min the next day, with the supernatant discarded. The precipitate was resuspended with 500 ul PBS and dispensed with 50 ul per tube, and stood at −80° C.

### Example 6: Construction of iPS Cells in which CAR was Stably Expressed

After the titer of the virus was determined, iPS was infected with the virus with MOI being 20, 0.25 ug/ml puromycin was added on day 3 after infection for screening cells for 3 days, and a cell line stably expressing CAR was obtained, which could be used for differentiation into macrophages in a later stage.

### Example 7: Modification of HLA-1

B2M gene is located on chromosome 15q21-22.2. B2M gene encodes an endogenous low molecular weight serum protein β2 microglobulin associated with the MHC-I β2 chain on the surface of almost all nucleated cells. We designed three gRNAs for the first exon of the B2M gene, which were ligated to the vector of PX458 containing Cas9 protein, the vector was then introduced, by electroporation, into the iPS cells in which CAR was stably expressed in Example 6, and the cells were screened with a medium containing puromycin. The screened cells were divided into two groups, one group was cultured normally and the other group was treated with 50 ng/ul IFN-γ for 48 h, while being normally cultured. The wild type of iPS cells in which CAR was stably expressed in example 6 was also divided into two groups, one group was normally cultured, and the other group was treated with 50 ng/ul IFN-γ for 48 h, while being normally cultured. These 4 groups of cells were then incubated separately with anti-B2M antibodies for flow cytometry, and the B2M knockout effect was examined on machine. The results showed that compared with the wild type of iPS cells stably expressing CAR in Example 6, for the B2M knockout cells, 48 h of IFN-γ treatment cannot induce B2M expression, indicating that the B2M gene had been knocked out from the cells.

### Example 8: Preparation of Macrophages Capable of Targeting Tumor Cells

### 1) Induction of iPS Cells Stably Expressing CAR to Form Embryoid Bodies (EB)

mTeSR1, DMEM/F12 and Versene are preheated to 15-25° C. for cell passage. Y27632 is a Rock kinase inhibitor and used at a concentration of 3 μM. The cells of Example 7 were induced:

a) washing the wells with 1 ml DPBS;

b) aspirating DPBS, adding 1 ml Versene containing Y27632, and incubating at 37° C. for 4 min;

c) dissociating with a pipette 1-2 times and taking the cells out (in general, better EBs will be formed if the cells are still in larger mass);

d) immediately transferring the cells to a centrifuge tube containing DMEM/F12 to dilute Versene at a ratio of 1:5-9; washing the wells once with 1 ml DMEM/F12, collecting the remaining cells and transferring the same to a test tube for centrifugation at 300×g for 5 min; and

e) resuspending the cells in the mTeSR1 medium containing Y27632 and placing the cells on an ultra-low attachment plate, the segregation ratio being 1-2:1 (90% of induced pluripotent stem cells per well).

2) Induction of Embryoid Bodies (EB) to Differentiate into Macrophages

step a) removing the mTeSR1 medium form the embryoid bodies in e) of 1), and incubating and culturing the embryoid bodies with the first medium (STEMdiff™ APEL™ 2, 10 ng/ml BMP4, 5 ng/ml bFGF) for 24 h on day 1, the embryoid bodies differentiating into mesoblastic cells;

step b) removing the first medium in step a), and incubating and culturing the mesoblastic cells with the second medium (STEMdiff™ APEL™ 2, 10 ng/ml BMP4, 5 ng/ml bFGF, 50 ng/ml VEGF and 100 ng/ml SCF) during days 2-7 after inoculation, during which the used second medium was replaced with a new second medium every other day, to obtain hematopoietic cells;

step c) removing the second medium in step b), and incubating and culturing the hematopoietic cells with the third medium (STEMdiff™ APEL™ 2, 10 ng/mlbFGF, 50 ng/ml VEGF, 50 ng/ml SCF, 10 ng/ml IGF1, 25 ng/ml IL-3, 50 ng/ml M-CSF and 50 ng/ml GM-CSF) during days 8-10 after inoculation, during which the used third medium was replaced with a new third medium every other day;

step d) removing the third medium in step c), inoculating the cells into a culture dish pre-coated with Matrigel (1 mg/ml) at a concentration of 20-25 cells/ml during days 11-20 after inoculation, and incubating and culturing the cells in step c) with the fourth medium (Stem Pro™-34, 10 ng/ml bFGF, 50 ng/ml VEGF, 50 ng/ml SCF, 10 ng/ml IGF1, 25 ng/ml IL-3, 50 ng/ml M-CSF and 50 ng/ml GM-CSF) to obtain myeloid cells;

step e) collecting the myeloid cells suspended in step d) from days 21-22 after inoculation, re-plating the myeloid cells in a culture dish pre-coated with a matrix gel, and incubating and culturing the myeloid cells with the fifth medium (StemPro™-34, 10 ng/ml bFGF, 50 ng/ml VEGF, 50 ng/ml SCF, 10 ng/ml IGF1, 25 ng/ml IL-3, 100 ng/ml M-CSF and 100 ng/ml GM-CSF), the myeloid cells differentiating into macrophages;

step f) removing the fifth medium in step e), and incubating the macrophages during days 23-28 after inoculation, using the sixth medium (StemPro™-34, 10 ng/ml bFGF, 50 ng/ml VEGF, 50 ng/ml SCF, 10 ng/ml IGF1, 100 ng/ml M-CSF and 100 ng/ml GM-CSF); and

step g) removing the sixth medium in step f), maintaining mature macrophages from day 29 after inoculation using the seventh medium (RPMI-1640, 10% w/w FBS, 100 ng/ml M-CSF, 100 ng/ml GM-CSF) or cryopreserving the cells.

A large number of high-quality and high-purity mature macrophages capable of targeting tumor cells were obtained by the method.

### Example 9: Flow Cytometry

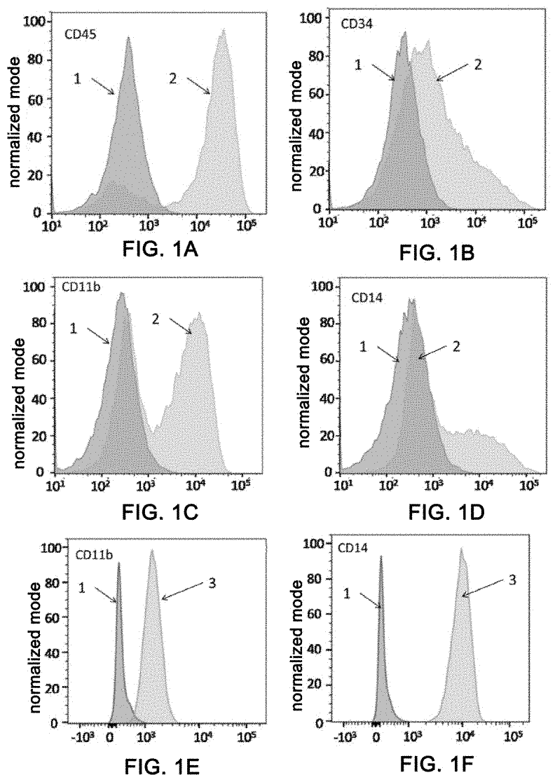

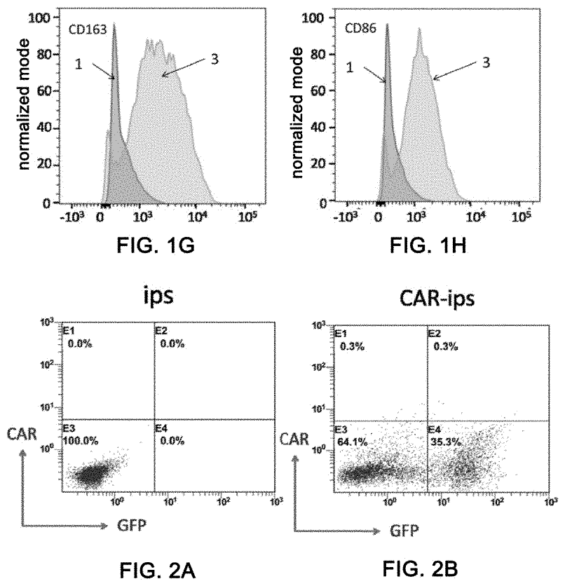

The cells of each stage obtained in Example 8 were subjected flow cytometry to detect the markers of relevant cells so as to evaluate the effect of directed differentiation. The results are shown in FIGS. 1A-1H. It should be noted that in FIGS. 1A-1H, 1 represents iPS cells, 2 represents myeloid cells on day 14, and 3 represents mature macrophages on day 45.

FIG. 1A shows the detection results of the marker CD45 for blood cells in myeloid cells on day 14, FIG. 1B shows the detection results of the marker CD34 of hematopoietic stem cells in myeloid cells on day 14, FIG. 1C shows the detection results of the marker CD11b for macrophages in myeloid cells on day 14, FIG. 1D shows the detection results of the marker CD14 of macrophages in myeloid cells on day 14, FIG. 1E shows the detection results of the marker CD11b for macrophages in mature macrophages on day 45, FIG. 1F shows the detection results of the marker CD14 for macrophages in mature macrophages on day 45, FIG. 1G shows the detection results of the marker CD163 for macrophages in mature macrophages on day 45, and FIG. 1H shows the detection results of the marker CD86 for macrophages in mature macrophages on day 45.

The results showed that the markers CD11 b and CD14 for macrophages appeared on day 14, the expression level of CD14 increased on day 45, and new markers CD86 and CD163 for macrophages appeared, indicating successful directed differentiation of pluripotent stem cells into mature macrophages.

### Example 10: Expression of Chimeric Antigen Receptors on the Surface of Macrophages

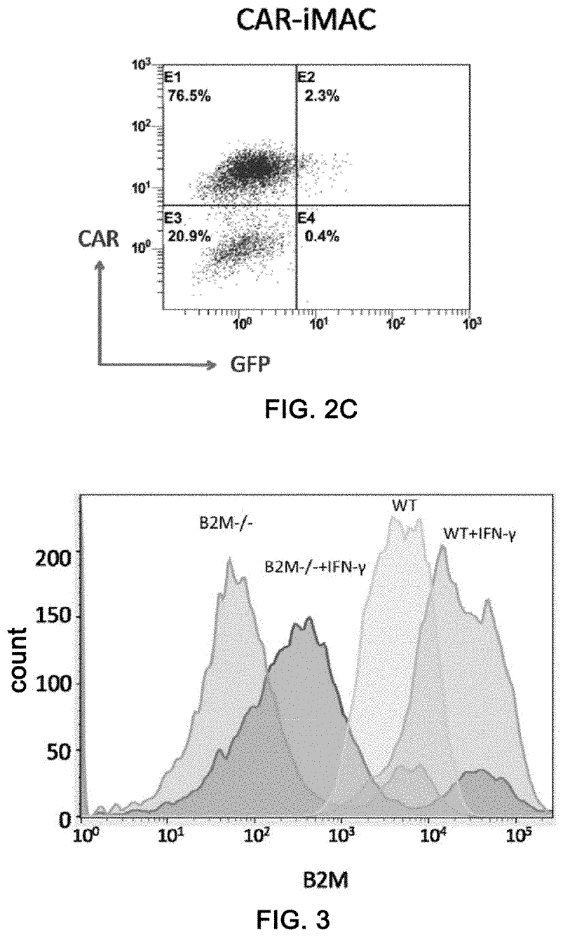

Whether the chimeric antigen receptors were expressed on the surface of the iPS cells and macrophages obtained by differentiation was identified by flow cytometry. Wild-type iPS cells and the iPS cells stably expressing chimeric antigen receptors in Example 6, as well as the macrophages (macrophages in Example 8) resulting therefrom by differentiation were centrifuged at 300 rcf for 5 min, with the supernatant removed, washed once with PBS, centrifuged repeatedly, incubated with anti-CAR flow cytometric antibodies for 15 min, centrifuged at 300 rcf for 5 min, with the supernatant removed, washed once with PBS, incubated with secondary antibodies for 10 min, centrifuged for 5 min, with the supernatant removed, and then washed once with PBS. The cells were then resuspended with PBS containing 0.1% BSA, and then detected on machine by flow cytometry. The results were shown in FIGS. 2A-2C, and it was found that CAR could be expressed on the surface of the macrophages.

### Example 11: Immunoassay of HLA-1 Deficiency

HLA-I (B2M) deficient pluripotent stem cells of Example 7 were divided into two groups, one group was cultured normally and the other group was treated with 50 ng/ul IFN-γ for 48 h. The wild type of iPS cells in which CAR was stably expressed in example 6 was also divided into two groups, one group was normally cultured, and the other group was treated with 50 ng/ul IFN-γ for 48 h. These 4 groups of cells were then incubated separately with anti-B2M flow cytometric antibodies, and the B2M knockout effect was detected by flow cytometry. The results were shown in FIG. 3, indicating that compared with the iPS cells in which CAR was stably expressed in Example 6, treatment of the B2M knockout cells with IFN-γ for 48 h cannot induce B2M expression, indicating that the B2M gene had been knocked out from the cells.

### Example 12: Assay for Specific Phagocytosis of Cancer Cells

K562 is an acute myeloid leukemia cell line that does not express CD19 antigen on its surface. A lentiviral vector expressing CD19 was transformed into K562 cells to construct a cell strain expressing CD19 on its cell surface. Raji is a cell line from B cell lymphoma, which expresses CD19 antigen on its cell surface. K562 cells, K562 cells stably expressing CD19, and Raji cells were infected with mcherry virus, and were sorted by flow cytometry 4-5 days later, followed by culturing and amplifying mcherry-positive stably transfected cell lines.

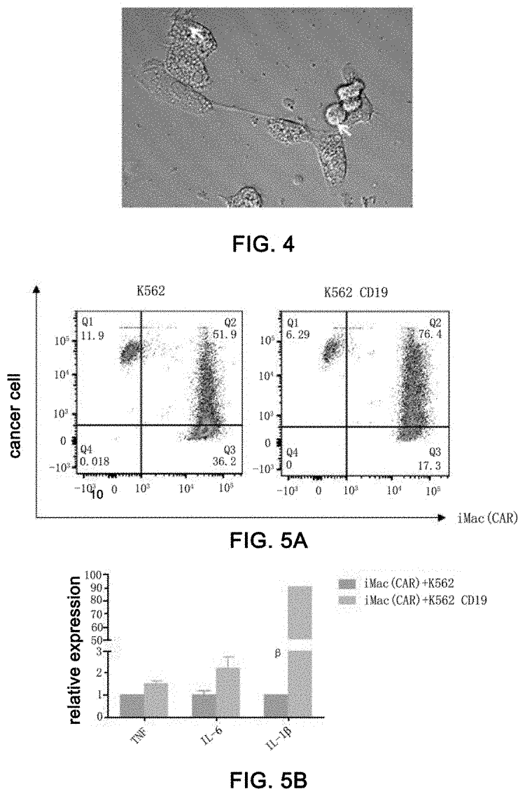

The macrophages obtained by differentiation in Example 8 were cultured respectively with the above-mentioned three mcherry stably transfected cell lines for 4 h, and then photographed with a confocal microscope, to count the macrophages phagocytizing cancer cells expressing mcherry. The results were shown in FIG. 4. The experiment results show that the macrophage provided by the present disclosure has the ability to phagocytize cancer cells, and also allows large-scale heterologous production application.

FIG. 5A shows culture of 2×10\hat{ }5 macrophages iMAC obtained by differentiation of iPS or ES cells overexpressing the chimeric antigen receptor CD19 CAR together with 4×10\hat{ }4 CD19 antigen-expressing K562 tumor cells and non-CD19 antigen-expressing K562 cells for 24 hours separately to detect the phagocytosis of the iMAC for tumor cells. iMAC and K562 cells were labeled with fluorescent dyes of different colors, and the double-labelled cells represented iMAC cells capable of phagocytizing tumor cells. The results showed that CD19 CAR iMAC had stronger phagocytosis on K562 cells expressing CD19 antigen. FIG. 5B shows, as the experiment of FIG. 5A, extraction of RNA from the macrophages that have been co-cultured with tumor cells, to detect the expression of cytokines (TNF, IL-6, and IL-1β). The macrophages co-cultured with K562 cells expressing CD19 antigen were significantly improved in TNF, IL-6 and IL-1β cytokines expression level.

Although the present disclosure has been illustrated and described with specific examples, it should be appreciated that many other changes and modifications may be made without departing from the spirit and scope of the present disclosure. Therefore, this means that all such variations and modifications falling within the scope of the present disclosure are included in the appended claims.

### INDUSTRIAL APPLICABILITY

The present disclosure provides a pluripotent stem cell-derived macrophage capable of targeting tumor cells, the macrophage containing a chimeric antigen receptor. The inventors have found that the CAR-T cell therapy has some technical defects in the treatment of tumors, i.e., due to the limitation of the microenvironment of a solid tumor, it is very difficult for CAR-T cells to enter the tumor, even if the CAR-T cells enter the tumor, the effect of killing tumor cells thereof is weakened due to the inhibition in the microenvironment. In view of the technical defects, the inventors have proposed another idea of tumor immunotherapy in which a chimeric antigen receptor is expressed in the macrophage. Compared with T cells, the macrophage has the advantages of being easier to enter the solid tumor and less likely to be inhibited by other types of cells, and therefore can play a better role in tumor immunotherapy. Since the expressed chimeric antigen receptor is located on the surface of the macrophage, the macrophage can accurately target tumor cells. Moreover, the inventors have found through experiments that the chimeric antigen receptor suitable for T cells is also suitable for the macrophage, that is, the application of the chimeric antigen receptor in the CAR-T cell therapy to the macrophage can realize expressing the chimeric antigen receptor on the surface of the macrophage, targeting tumor cells and activating the macrophage to phagocytize tumor cells. Therefore, the discovery of using a chimeric antigen receptor to modify a macrophage provides a new idea and technical means for solid tumor immunotherapy, which is of great significance for tumor immunotherapy.

The present disclosure provides a preparation method of the macrophage capable of targeting tumor cells, which provides a whole new idea for tumor immunotherapy.

What is claimed is:

1. A macrophage capable of targeting tumor cells, wherein the macrophage comprises a chimeric antigen receptor.

2. The macrophage according to claim 1, wherein the macrophage is an HLA-I deficient macrophage.

3. The macrophage according to claim 2, wherein the macrophage is a B2M gene-deficient macrophage.

4. The macrophage according to claim 1, wherein the macrophage is obtained by directed differentiation of a pluripotent stem cell containing a gene encoding the chimeric antigen receptor.

5. The macrophage according to claim 4, wherein the pluripotent stem cell is an HLA-I deficient pluripotent stem cell.

6. The macrophage according to claim 5, wherein the pluripotent stem cell is a B2M gene-deficient pluripotent stem cell.

7. The macrophage according to claim 4, wherein the pluripotent stem cell comprises an induced pluripotent stem cell and/or an embryonic stem cell.

8. The macrophage according to claim 1, wherein the chimeric antigen receptor comprises an extracellular antigen binding region, a transmembrane region, a costimulatory domain, and an intracellular signal transduction region.

9. The macrophage according to claim 8, wherein:
the extracellular antigen binding region comprises an sc-Fv, Fab, scFab, or scIgG antibody fragment; and/or
the transmembrane region comprises at least one of CD3ζ, CD4, CD8 and CD28; and/or
the costimulatory domain comprises at least one ligand specifically binding to CD27, CD28, CD137, OX40, CD30, CD40, PD-1, LFA-1, CD2, CD7, Lck, DAP10, ICOS, LIGHT, NKG2C, B7-H3, or CD3ζ; and/or
the intracellular signal transduction region comprises at least one of CD3ζ, FcεRlγ, PKCθ and ZAP70.

10. The macrophage according to claim 8, wherein the chimeric antigen receptor further comprises a reporter gene.

11. The macrophage according to claim 8, wherein the extracellular antigen binding region specifically binds to at least one of: CD19, CD20, CD22, CD30, GD2, HER2, CAIX, CD171, Mesothelin, Claudin 18.2, LMP1, EGFR, Muc1, GPC3, EphA2, EpCAM, MG7, CSR, α-fetoprotein (AFP), α-actinin-4, A3, an antigen specific to A33 antibody, ART-4, B7, Ba 733, BAGE, BrE3 antigen, CA125, CAMEL, CAP-1, carbonic anhydrase IX, CASP-8/m, CCL19, CCL21, CD1, CD1a, CD2, CD3, CD4, CD5, CD8, CD11A, CD14, CD15, CD16, CD18, CD21, CD23, CD25, CD29, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD126, CD132, CD133, CD138, CD147, CD154, CDC27, CDK-4/m, CDKN2A, CTLA4, CXCR4, CXCR7, CXCL12, HIF-1α, colon specific antigen p, CEACAM-5, CEACAM-6, c-Met, DAM, EGFRvIII, EGP-1, EGP-2, ELF2-M, Ep-CAM, a fibroblast growth factor, Flt-1, Flt-3, a folate receptor, G250 antigen, GAGE, gp100, GRO-β, HLA-DR, HM1.24, human chorionic gonadotropin and its subunits, HMGB-1, hypoxia-inducible factor, HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor 1, KC4 antigen, KS-1 antigen, KS1-4, Le-Y, LDR/FUT, macrophage migration inhibitory factor, MAGE, MAGE-3, MART1, MART-2, NY-ESO-1, TRAG-3, mCRP, MCP-1, MIP-1A, MIP-1B, MIF, MUC2, MUC3, MUC4, MUC5ac, MUC13, MUC16, MUM-1/2, MUM-3, NCA66, NCA95, NCA90, pancreatic cancer mucin, a PD1 receptor, a placental growth factor, p53, PLAGL2, prostatic acid phosphatase, PSA, PRAME, PSMA, PIGF, ILGF, ILGF-1R, IL-6, IL-25, RS5, RANTES, T101, SAGE, S100, survivin, survivin-2B, TAC, TAG-72, tenascin, a TRAIL receptor, TNF-α, Tn antigen, Thomsen-Friedenreich antigen, a tumor necrosis antigen, VEGFR, ED-B fibronectin, WT-1, 17-1A antigen, complement factors C3, C3a, C3b, C5a and C5, an angiogenesis marker, bc1-2, bc1-6, and Kras.

12. The macrophage according to claim 8, wherein the extracellular antigen binding region specifically binds to CD19.

13. A preparation method of the macrophage according to claim 1, comprising allowing a gene encoding a chimeric antigen receptor to be expressed on the macrophage to obtain the macrophage capable of targeting tumor cells.

14. The preparation method according to claim 13, wherein the preparation method further comprises at least one of:
a step of preparing an HLA-I gene-deficient macrophage; and
a step of preparing a B2M gene-deficient macrophage.

15. The preparation method according to claim 13, wherein the preparation method comprises directed differentiation of a pluripotent stem cell into a macrophage capable of targeting tumor cells, the pluripotent stem cell containing a gene encoding a chimeric antigen receptor.

16. The preparation method according to claim 13, wherein the pluripotent stem cell is an HLA-I deficient and/or B2M gene-deficient pluripotent stem cell.

17. The preparation method according to claim 15, wherein the directed differentiation comprises the steps of:
placing an embryoid body resulting from induced differentiation of a pluripotent stem cell in a first medium for a first stage culture, and
performing a second stage culture in a second medium, a third stage culture in a third medium, a fourth stage culture in a fourth medium, a fifth stage culture in a fifth medium, a sixth stage culture in a sixth medium, and a seventh stage culture in a seventh medium, sequentially,
wherein the first stage is days 0-1 after inoculation, the second stage is days 2-7 after inoculation, the third stage is days 8-10 after inoculation, the fourth stage is days 10-20 after inoculation, the fifth stage is days 20-22 after inoculation, the sixth stage is days 22-28 after inoculation, and the seventh stage is day 29 after inoculation.

18. The preparation method according to claim 17, wherein the first medium comprises a first basal medium and first cytokines comprising BMP4 and bFGF;
the second medium comprises the first basal medium and second cytokines comprising BMP4, bFGF, VEGF and SCF;
the third medium comprises the first basal medium and third cytokines comprising bFGF, VEGF, SCF, IGF1, IL-3, M-CSF and GM-CSF;
the fourth medium comprises a second basal medium and the third cytokines;
the fifth medium comprises the second basal medium and fourth cytokines comprising bFGF, VEGF, SCF, IGF1, IL-3, M-CSF and GM-CSF;
the sixth medium comprises the second basal medium and fifth cytokines comprising bFGF, VEGF, SCF, IGF1, M-CSF and GM-CSF;
the seventh medium comprises a third basal medium, sixth cytokines and FBS, the sixth cytokines comprising M-CSF and GM-CSF;
wherein the first basal medium and the second basal medium are serum-free mediums; and the third basal medium is a serum-containing medium.

19. A method for preventing or treating a tumor, comprising administering the macrophage capable of targeting tumor cells according to claim 1 to a subject in need thereof.

20. The method according to claim 19, wherein the tumor includes at least one of acute lymphoblastic leukemia, acute myelogenous leukemia,
cholangiocarcinoma, breast cancer, cervical cancer, chronic lymphocytic leukemia, chronic myelogenous leukemia, colorectal cancer, endometrial cancer, esophageal cancer, gastric cancer, head and neck cancer, Hodgkin's lymphoma, lung cancer, medullary thyroid carcinoma, non-Hodgkin's lymphoma, multiple myeloma, kidney cancer, ovarian cancer, pancreatic cancer, neuroglioma, melanoma, liver cancer, prostate cancer and urinary bladder cancer.
